(19) United States
(12) Patent Application Publication
(10) Pub. No.: US 2026/0315554 A1
(43) Pub. Date: Oct. 15, 2026

(54) COMPOSITIONS AND METHODS OF MAKING AND USE THEREOF

(71) Applicant: Ohio State Innovation Foundation, Columbus, OH (US)

(72) Inventors: Sheila JACOBI, Columbus, OH (US); Rafael JIMENEZ-FLORES, Columbus, OH (US); Erica KOSMERL, Columbus, OH (US); Celeste MILLER, New Franklin, OH (US); Rebecca BROWN, Columbus, OH (US)

(21) Appl. No.: 19/485,380

(22) PCT Filed: May 17, 2024
(86) PCT No.: PCT/US2024/029831

§ 371 Date: Nov. 18, 2025

Related U.S. Application Data
(60) Provisional application No. 63/467,688, filed on May 19, 2023.

(51) Int. Cl. A61K 35/747; A23K 10/18; A23K 10/20; A23K 20/142; A61K 31/198; A61K 35/20

(52) U.S. Cl. CPC A61K 35/747; A23K 10/18; A23K 10/20; A23K 20/142; A61K 31/198; A61K 35/20

(57) ABSTRACT

Disclosed herein are compositions and methods of making and use thereof. For example, disclosed herein are compositions comprising a probiotic lactic acid bacteria and milk fat globule membrane. Also disclosed herein are products comprising any of the compositions disclosed herein, such as a nutritional product or supplement, a food product (e.g., a functional food product), or a combination thereof. For example, the composition or product can be for the treatment, prevention, or amelioration of at least one symptom of depression, stress, anxiety, or a depressive or anxiety-related disorder in a subject in need thereof. Also disclosed herein are methods of use and methods of making of any of the compositions or any of the products disclosed herein.

### CROSS-REFERENCE TO RELATED APPLICATIONS

This application claims the benefit of priority to U.S. Provisional Application No. 63/467,688 filed May 19, 2023, which is hereby incorporated herein by reference in its entirety.

### BACKGROUND

Compositions with improved properties are needed. For example, current market trends demonstrate the demand by consumers for functional foods that deliver benefits beyond their nutritional value, with specific emphasis on calming products that promote emotional and mental well-being and reduce stress and anxiety. The compositions and methods discussed herein addresses these and other needs.

### SUMMARY

In accordance with the purposes of the disclosed compositions and methods as embodied and broadly described herein, the disclosed subject matter relates to compositions and methods of making and use thereof.

For example, disclosed herein are compositions comprising a probiotic lactic acid bacteria and a milk fat globule membrane.

In some examples, the probiotic lactic acid bacteria comprises Pediococcus acidilactici, Lactobacillus helveticus, Limosilactobacillus reuteri, Lacticaseibacillus rhamnosus, or a combination thereof. In some examples, the probiotic lactic acid bacteria comprises Pediococcus acidilactici and Lactobacillus helveticus.

In some examples, the milk fat globule protein is derived from a milk. In some examples, the milk is obtained from a mammal, such as milk obtained from a cow, goat, horse, donkey, buffalo, camel, or sheep. In some examples, the milk fat globule protein is a whey-derived milk fat globule membrane.

In some examples, the composition further comprises any suitable additives, carriers, additional therapeutic agents, bioavailability enhancers, side-effect suppressing components, diluents, buffers, flavoring agents, binders, preservatives or other ingredients that are not detrimental to the efficacy of the composition.

In some examples, the composition further comprises an amino acid precursor (e.g., L-Tryptophan).

Also disclosed herein are products comprising any of the compositions disclosed herein. In some examples, the product comprises a nutritional product or supplement. In some examples, the product comprises a food product, such as a functional food product. In some examples, the product comprises any product that normally contains milk or milk solids or milk protein. In some examples, the product comprises a dairy product, such as yogurt, cheese, ice cream, pudding, flavored milk, or butter. In some examples, the product comprises a non-fermented or a fermented food (e.g., yogurt, cheese, cultured dairy products). In some examples, the product comprises a beverage. In some examples, the product comprises a dessert product, such as a frozen desserts (e.g., ice cream). In some examples, the product comprises an animal food (e.g., pet food, livestock food, and/or laboratory animal food).

In some examples, the composition or product is for the treatment, prevention, or amelioration of at least one symptom of depression, stress, anxiety, or a depressive or anxiety-related disorder in a subject in need thereof.

The composition of any one of claims 1-8 or the product of any one of claims 9-17, wherein the composition or product is for treating serotonin deficiency and/or a disease or disorder related to serotonin deficiency in a subject in need thereof.

Also disclosed herein are methods of use of any of the compositions or any of the products disclosed herein.

In some examples, the method comprises the treatment, prevention, or amelioration of at least one symptom of depression, stress, anxiety, or a depressive or anxiety-related disorder in a subject in need thereof. In some examples, the method comprises treating serotonin deficiency and/or a disease or disorder related to serotonin deficiency in a subject in need thereof.

In some examples, when the composition and/or product is consumed by a subject, the composition and/or product enhances neurotransmitter production in the gastrointestinal tract of the subject. In some examples, the neurotransmitter comprises serotonin, dopamine, GABA, or a combination thereof. In some examples, the neurotransmitter comprises serotonin.

In some examples, the method results in improved mood, improved gut health, improved satiety, or a combination thereof in the subject.

In some examples, the subject experiences high stress, such as military personnel and medical professionals.

In some examples, the subject comprises livestock, a laboratory animal, or a combination thereof.

In some examples, the subject comprises pigs.

Also disclosed herein are methods of making any of the compositions or any of the products disclosed herein.

Additional advantages of the disclosed compositions and methods will be set forth in part in the description which follows, and in part will be obvious from the description. The advantages of the disclosed compositions and methods will be realized and attained by means of the elements and combinations particularly pointed out in the appended claims. It is to be understood that both the foregoing general description and the following detailed description are exemplary and explanatory only and are not restrictive of the disclosed compositions and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1A. Growth curves of L. helveticus 4A in the minimal media casein glucose broth (CGB) in the presence or absence of 0.5% milk fat globule membrane (MFGM) under aerobic conditions at 37° C.

FIG. 1B. Growth curves of P. acidilactici 3A in the minimal media casein glucose broth (CGB) in the presence or absence of 0.5% milk fat globule membrane (MFGM) under aerobic conditions at 37° C.

FIG. 1C. Growth curves of L. reuteri 48 in the minimal media casein glucose broth (CGB) in the presence or absence of 0.5% milk fat globule membrane (MFGM) under aerobic conditions at 37° C.

FIG. 1D. Growth curves of L. rhamnosus 69 in the minimal media casein glucose broth (CGB) in the presence or absence of 0.5% milk fat globule membrane (MFGM) under aerobic conditions at 37° C.

FIG. 2. Increased serotonin (5-HT) production by probiotic lactic acid bacteria in the presence of 0.5% milk fat globule membrane (MFGM) and the amino acid precursor, L-Trp, using the minimum bacterial culture media casein glucose broth (CGB). Measurements were obtained using the colorimetric Ehrlich's reagent assay using absorbance at 625 nm on a spectrophotometric plate reader. Data are represented as mean±SD with significance values using a Student's t-test (n=3).

FIG. 3. Serotonin (5-HT) production by 10 strains of lactic acid bacteria chosen from a screening of 137 strains in the early stationary phase in the presence of 0.5% milk fat globule membrane (MFGM) and the amino acid precursor, L-Trp, using the minimum bacterial culture media casein glucose broth (CGB). Measurements were obtained using the colorimetric Ehrlich's reagent assay using absorbance at 625 nm on a spectrophotometric plate reader. Data are represented as mean±SD with significance values using a Student's t-test (n=6).

FIG. 4. Serotonin (5-HT) production by 10 strains of lactic acid bacteria chosen from a screening of 137 strains in the late stationary phase in the presence of 0.5% milk fat globule membrane (MFGM) and the amino acid precursor, L-Trp, using the minimum bacterial culture media casein glucose broth (CGB). Measurements were obtained using the colorimetric Ehrlich's reagent assay using absorbance at 625 nm on a spectrophotometric plate reader. Data are represented as mean±SD with significance values using a Student's t-test (n=6).

FIG. 5. Exopolysaccharide (EPS) production by 10 strains of lactic acid bacteria chosen from a screening of 137 strains in the late stationary phase in the presence of 0.5% milk fat globule membrane (MFGM) and the amino acid precursor, L-Trp, using the minimum bacterial culture media casein glucose broth (CGB). Measurements were obtained using the colorimetric phenol sulfuric total sugar assay using absorbance at 480 nm and subtraction of the colorimetric DNSA reducing sugar assay using an absorbance at 540 nm on a spectrophotometric plate reader. Data are represented as mean of the reducing sugars subtracted from the total sugars with significant values using a Student's t-test (n=6).

FIG. 6. Nutrition facts label for 2-pack container of pudding product.

FIG. 7. Jar container used for sensory analysis of the pudding product.

FIG. 8. Process flow diagram for the manufacture of the pudding product.

FIG. 9. Product is packaged in a milk bottle-shaped glass jar containing dark chocolate pudding decorated with a white chocolate topping containing live and active cultures.

FIG. 10. Alternative photograph of product packaged in a milk bottle-shaped glass jar containing dark chocolate pudding decorated with a white chocolate topping containing live and active cultures.

FIG. 11. Front of product packaged in a 2-pack format using a recyclable paper sleeve.

FIG. 12. Back of product packaged in a 2-pack format using a recyclable paper sleeve.

FIG. 13. Top of product packaged in a 2-pack format using a recyclable paper sleeve.

FIG. 14. Packaging mock-up to be distributed and sold in a 2-pack format.

FIG. 15. Results of product's hedonic evaluation concept liking, overall product liking, appearance, and texture using a 9-point scale (n=32).

FIG. 16. Results of the product's Just-About-Right evaluation of sweetness and chocolate flavor using a 5-point scale (n=32).

FIG. 17. Participants that agreed with the statement “The pudding is smooth” regarding product's sensory evaluation using a 7-point Likert scale (n=32).

FIG. 18. Results of the product performance based on the expectations generated by the provided product concept (n=32).

FIG. 19. Panelists' purchase intent for product 2-pack (net content: 200 g of product) (n=32).

### DETAILED DESCRIPTION

The compositions and methods described herein may be understood more readily by reference to the following detailed description of specific aspects of the disclosed subject matter and the Examples included therein.

Before the present compositions and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Values can be expressed herein as an “average” value. “Average” generally refers to the statistical mean value.

By “substantially” is meant within 5%, e.g., within 4%, 3%, 2%, or 1%.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

“Whey” or “liquid whey” is a collective term referring to the serum or watery part of milk that remains after the manufacture of cheese or yogurt or other milk byproducts. The milk may be from one or more ruminants, including domesticated ruminants, e.g. cows, sheep, goats, yaks, water buffalo, bison, Alpaca, Llama, horses or camels. Whey derived from bovine milk, i.e. milk from cows, is presently preferred. In the present context, the term “native acid whey” (also known as sour whey) relates to the whey liquid, which is obtained during chemical or biological acidification of milk products, which for example take place in the production of cottage cheese or quark, yogurt, or in the production of casein/caseinates. The acid whey used in the invention may be native acid whey, e.g. filtered to remove particles of coagulated casein and fat. The acid whey is preferably in the form of a liquid, e.g. an aqueous solution.

In the context of the present invention, the term “dry weight” of a substance relates to the weight of the substance if it had been dried to a water content of 4% (w/w) water.

In the context of the present invention, the term “sweet whey” relates to the whey liquid, which is obtained during rennet-based coagulation of milk products, which for example take place in the production of yellow cheese. The term “whey protein powder” relates to the product obtained by drying the liquid sweet whey. In the present context, the expressions “whey protein concentrate (WPC)” relates to the dry portion of liquid whey obtained by the removal of sufficient non-protein constituents from whey so that the dry product contains not less than 25% protein. The term “whey protein isolate (WPI)” is for the dry portion of whey obtained by the removal of non-protein constituents from whey so that the dry whey product contains more than 90% whey protein on DS % basis. In some preferred embodiments of the invention the used whey powder concentrate and/or whey powder isolate is derived from sweet whey.

“Whey protein” is the name for a collection of globular proteins that can be isolated from liquid whey. It is typically a mixture of beta-lactoglobulin (~65%), alpha-lactalbumin (~25%), and serum albumin (~8%), which are soluble in their native forms, independent of pH. Whey protein derived from sweet whey additionally contains caseino-glycomacropeptide (cGMP).

The terms “casein” and “casein micelle” are used herein in their traditional meaning and relate to the most predominant phosphoprotein found in milk and cheese. Casein micelles have properties that make them a unique system for protein blending, including: ⋅ swelling at low temperatures, swelling at alkaline pH, and dissociating with the addition of calcium chelating agents.

By “globular protein” is meant any protein obtained from a non-dairy plant or animal source. The globular proteins disclosed herein can be from plant sources. The globular proteins can be soluble or dispersible plant proteins. For example, the plant source can be chia, rice, coconut, almond, soy, oat, hemp, peanut, pea, cashew, quinoa, or flax. The plant source can also be a seed, such as grape seeds or other seeds used in brewing or distilling. The globular proteins can also be obtained from animal sources, such as insects. Examples include, but are not limited to, beetles, caterpillars, bees, wasps, grasshoppers, locusts, crickets, cicadas, leafhoppers and bugs, termites, dragonflies, and flies.

By “dairy product” is meant any type of food produced from or containing the milk of mammals. They are primarily produced from mammals such as cattle, water buffaloes, goats, sheep, donkeys, camels, and humans. Dairy products include food items, such as yogurt, cheese, and butter. The dairy product can be, for example, milk, such as skim milk.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), birds, and insects. “Subject” can also include a mammal, such as a primate or a human. Thus, the subject can be a human or veterinary patient. The term “patient” refers to a subject under the treatment of a clinician, e.g., physician.

As used herein, antimicrobials include, for example, antibacterials, antifungals, and antivirals. As used herein, “antimicrobial” refers to the ability to treat or control (e.g., reduce, prevent, treat, or eliminate) the growth of a microbe at any concentration. Similarly, the terms “antibacterial,” “antifungal,” and “antiviral” refer to the ability to treat or control the growth of bacteria, fungi, and viruses at any concentration, respectively.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, “reduce” or other forms of the word, such as “reducing” or “reduction,” refers to lowering of an event or characteristic (e.g., microbe population/infection). It is understood that the reduction is typically in relation to some standard or expected value, in other words it is relative, but that it is not always necessary for the standard or relative value to be referred to. For example, “reducing microbial infection” means reducing the spread of a microbial infection relative to a standard or a control.

As used herein, “prevent” or other forms of the word, such as “preventing” or “prevention,” refers to stopping a particular event or characteristic, stabilizing or delaying the development or progression of a particular event or characteristic, or minimizing the chances that a particular event or characteristic will occur. “Prevent” does not require comparison to a control as it is typically more absolute than, for example, “reduce.” As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

As used herein, “treat” or other forms of the word, such as “treated” or “treatment,” refers to administration of a composition or performing a method in order to reduce, prevent, inhibit, or eliminate a particular characteristic or event (e.g., microbe growth or survival). The term “control” is used synonymously with the term “treat.”

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. By way of example, in the context of microbial infections, “treating,” “treat,” and “treatment” as used herein, refers to partially or completely inhibiting or reducing the microbial infections which the subject is suffering. In one embodiment, this term refers to an action that occurs while a patient is suffering from, or is diagnosed with, the microbial infections, which reduces the severity of the condition, or retards or slows the progression of the condition. Treatment need not result in a complete cure of the condition; partial inhibition or reduction of the microbial infections is encompassed by this term.

The term “therapeutically effective amount” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “delivery” encompasses both local and systemic delivery. For example, delivery of mRNA encompasses situations in which an mRNA is delivered to a target tissue and the encoded protein or peptide is expressed and retained within the target tissue (also referred to as “local distribution” or “local delivery”), and situations in which an mRNA is delivered to a target tissue and the encoded protein or peptide is expressed and secreted into patient's circulation system (e.g., serum) and systematically distributed and taken up by other tissues (also referred to as “systemic distribution” or “systemic delivery).

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

### Compostions and Methods of Making and Use Thereof

Disclosed herein are compositions and methods of making and use thereof.

For example, disclosed herein are compositions comprising a probiotic lactic acid bacteria and milk fat globule membrane.

The probiotic lactic acid bacteria can be used or be present as specially selected strains as a culture concentrate or as part of a multi-strain blend, optionally with a variety of excipients.

Also contemplated by the present disclosure are variants of the probiotic lactic acid bacteria strains described herein. As used herein, the term “variant” refers to both naturally occurring and specifically developed variants or mutants of the probiotic lactic acid bacteria strains disclosed and exemplified herein. Variants may or may not have the same identifying biological characteristics of the specific strains exemplified herein, provided they share similar advantageous properties in terms of their ability to be used as probiotic strains.

Also encompassed by the term “variant” as used herein are probiotic lactic acid bacteria strains phylogenetically closely related to strains disclosed herein and strains possessing substantial sequence identity with the strains disclosed herein at one or more phylogenetically informative markers such as rRNA genes, elongation and initiation factor genes, RNA polymerase subunit genes, DNA gyrase genes, heat shock protein genes and recA genes.

The probiotic lactic acid bacteria strains to be employed in accordance with the present disclosure may be cultured according to any suitable method.

In some examples, the probiotic lactic acid bacteria comprises Pediococcus acidilactici, Lactobacillus helveticus, Limosilactobacillus reuteri, Lacticaseibacillus rhamnosus, or a combination thereof. In some examples, the composition comprises Pediococcus acidilactici and Lactobacillus helveticus.

In some examples, the milk fat globule protein is derived from a milk. In some examples, the milk is obtained from a mammal, such as milk obtained from a cow, goat, horse, donkey, buffalo, camel, or sheep. The milk fat globule membrane can, for example, be isolated from dairy waste streams, such as acid whey and buttermilk for example as described in WO2020/198342. In some examples, the milk fat globule protein is a whey-derived milk fat globule membrane.

In some examples, the composition further comprises any suitable additives, carriers, additional therapeutic agents, bioavailability enhancers, side-effect suppressing components, diluents, buffers, flavoring agents, binders, preservatives or other ingredients that are not detrimental to the efficacy of the composition. In some examples, the composition further comprises an amino acid precursor (e.g., L-Tryptophan).

Also disclosed herein are products comprising any of the compositions disclosed herein.

The compositions can, for example, be conveniently incorporated in a variety of food and/or beverage products, nutraceutical products, probiotic supplements, food additives, pharmaceuticals and over-the-counter formulations. The food or food additive can be a solid form such as a powder, or a liquid form. Specific examples of the types of beverages or foods include, but are not limited to water-based, milk-based, yogurt-based, other dairy-based, milk-substitute based such as soy milk or oat milk, or juice-based beverages, water, soft drinks, carbonated drinks, and nutritional beverages, (including a concentrated stock solution of a beverage and a dry powder for preparation of such a beverage); baked products such as crackers, breads, muffins, rolls, bagels, biscuits, cereals, bars such as muesli bars, health food bars and the like, dressings, sauces, custards, yoghurts, puddings, pre-packaged frozen meals, soups and confectioneries.

The products can additionally include any suitable additives, carriers, additional therapeutic agents, bioavailability enhancers, side-effect suppressing components, diluents, buffers, flavoring agents, binders, preservatives or other ingredients that are not detrimental to the efficacy of the composition.

For example, the product comprises a nutritional product or supplement. In some examples, the product comprises a food product, such as a functional food product. In some examples, the product comprises any product that normally contains milk or milk solids or milk protein. In some examples, the product comprises a dairy product, such as yogurt, cheese, ice cream, pudding, flavored milk, or butter. In some examples, the product comprises a non-fermented or a fermented food (e.g., yogurt, cheese, cultured dairy products). In some examples, the product comprises a beverage. In some examples, the product comprises a dessert product, such as a frozen desserts (e.g., ice cream). In some examples, the product comprises an animal food (e.g., pet food, livestock food, and/or laboratory animal food).

In some examples, the composition or product is for the treatment, prevention, or amelioration of at least one symptom of depression, stress, anxiety, or a depressive or anxiety-related disorder in a subject in need thereof. In some examples, the composition or product is for treating serotonin deficiency and/or a disease or disorder related to serotonin deficiency in a subject in need thereof.

Also disclosed herein are methods of use of any of the compositions or products disclosed herein. In some examples, the method comprises the treatment, prevention, or amelioration of at least one symptom of depression, stress, anxiety, or a depressive or anxiety-related disorder in a subject in need thereof. In some examples, the method comprises treating serotonin deficiency and/or a disease or disorder related to serotonin deficiency in a subject in need thereof.

The depressive disorder can, for example, be major depressive disorder, dysthymic disorder, seasonal affective disorder, mood disorder, mixed anxiety-depressive disorder or bipolar disorder. The depression can, for example, be mild depression, moderate depression, severe depression or postpartum depression. The anxiety-related disorder can, for example, be generalized anxiety disorder, mixed anxiety-depressive disorder or post-traumatic stress disorder.

In some examples, when the composition and/or product is consumed by a subject, the composition and/or product enhances neurotransmitter production in the gastrointestinal tract of the subject. Examples of neurotransmitters include, but are not limited to, serotonin, dopamine, GABA, and combinations thereof. In some examples, the neurotransmitter comprises serotonin.

In some examples, the method results in improved mood, improved gut health, improved satiety, or a combination thereof in the subject.

In some examples, the subject experiences high stress, such as military personnel, medical professionals, and livestock.

In some examples, the subject comprises livestock, a laboratory animal, or a combination thereof. In some examples, the subject comprises pigs.

Also disclosed herein are methods of making any of the compositions or products disclosed herein. For example, the compositions and products can be manufactured by those skilled in the art using known techniques and processes.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the devices and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1

Described herein are combinations of lactic acid bacteria and milk fat globule membrane in food or nutritional supplements as means to generate high production of neurotransmitters in the intestine.

Current market trends demonstrate the demand by consumers for functional foods that deliver benefits beyond their nutritional value, with specific emphasis on calming products that promote emotional and mental well-being and reduce stress and anxiety. The current work highlights a formulation containing milk fat globule membrane (MFGM) and probiotic lactic acid bacteria that promotes neurotransmitter production in the gastrointestinal tract (GIT).

A milk fat globule membrane-enriched ingredient can, for example, be isolated from dairy waste streams, such as acid whey and buttermilk for example as described in WO2020/198342. Lactic acid bacteria isolated from various dairy products have been collected and screened for their probiotic properties, genomic characteristics, and enzymatic activities. Four strains have been identified that display probiotic properties (acidic pH and bile salt tolerance, adhesion to intestinal cell culture, etc.) and possess the genes required for neurotransmitter synthesis (i.e., serotonin, dopamine, GABA) based on their complete genome sequences: Pediococcus acidilactici OSU-PECh 3A, Lactobacillus helveticus OSU-PECh 4A, Limosilactobacillus reuteri OSU-PECh 48, and Lacticaseibacillus rhamnosus OSU-PECh 69.

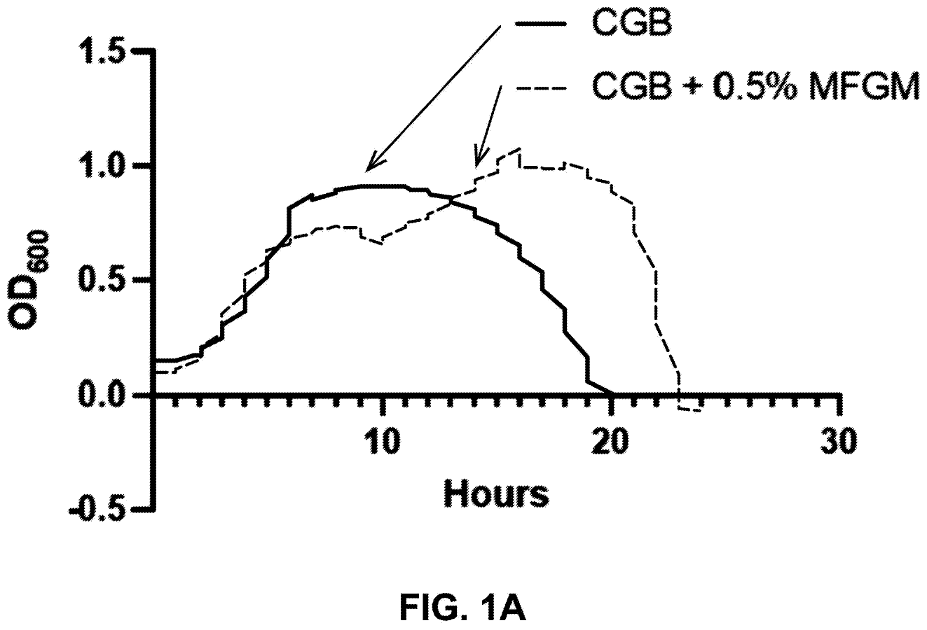

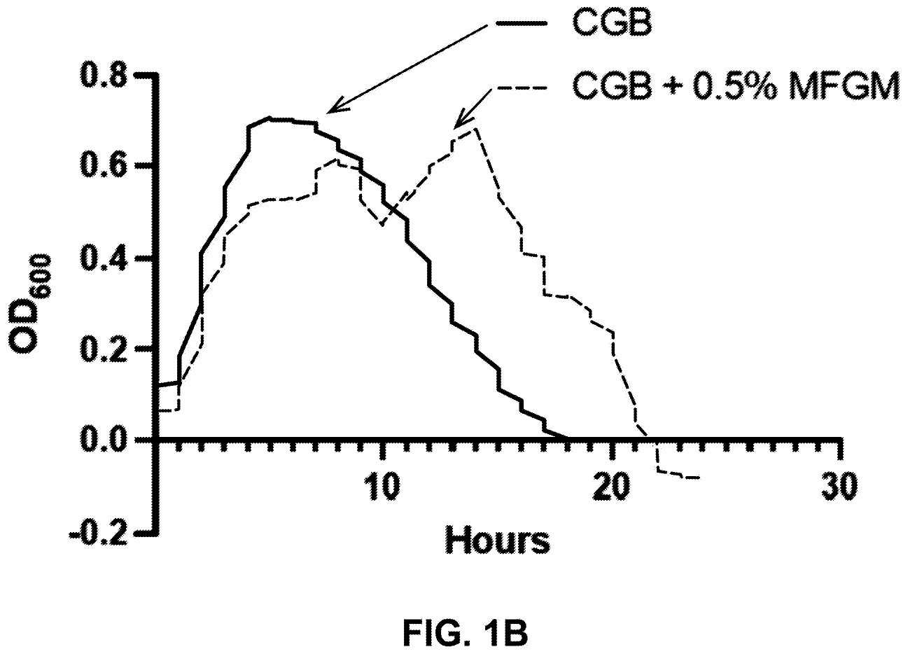

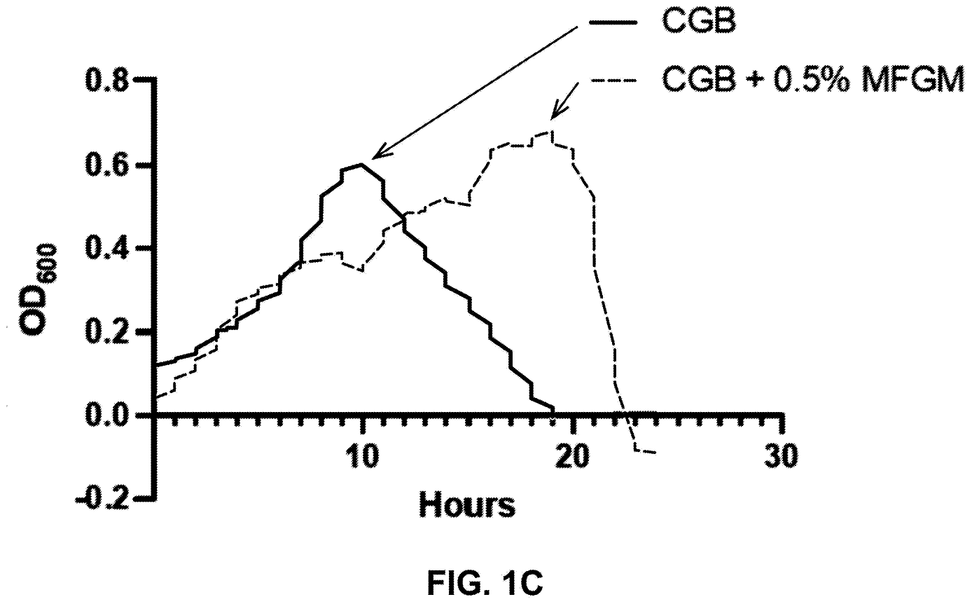

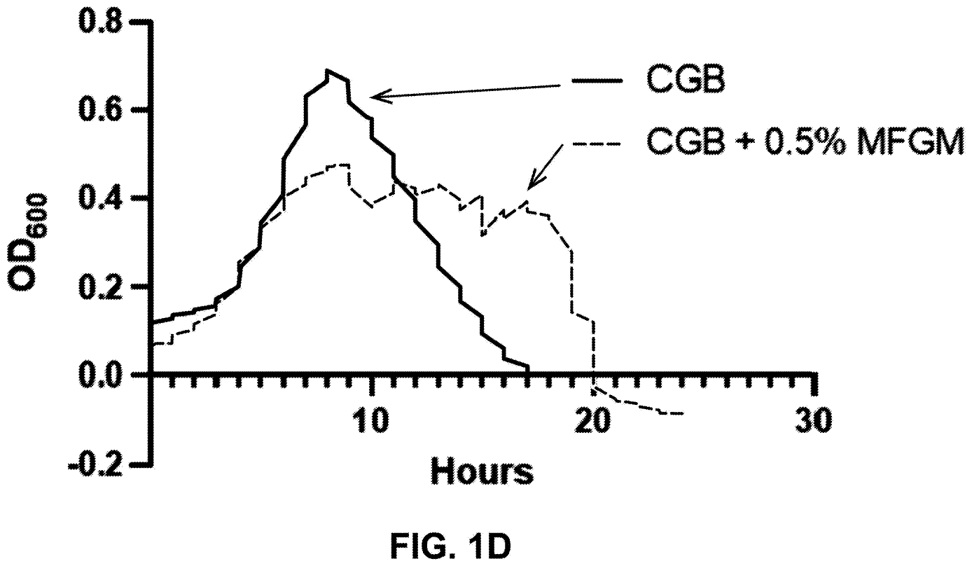

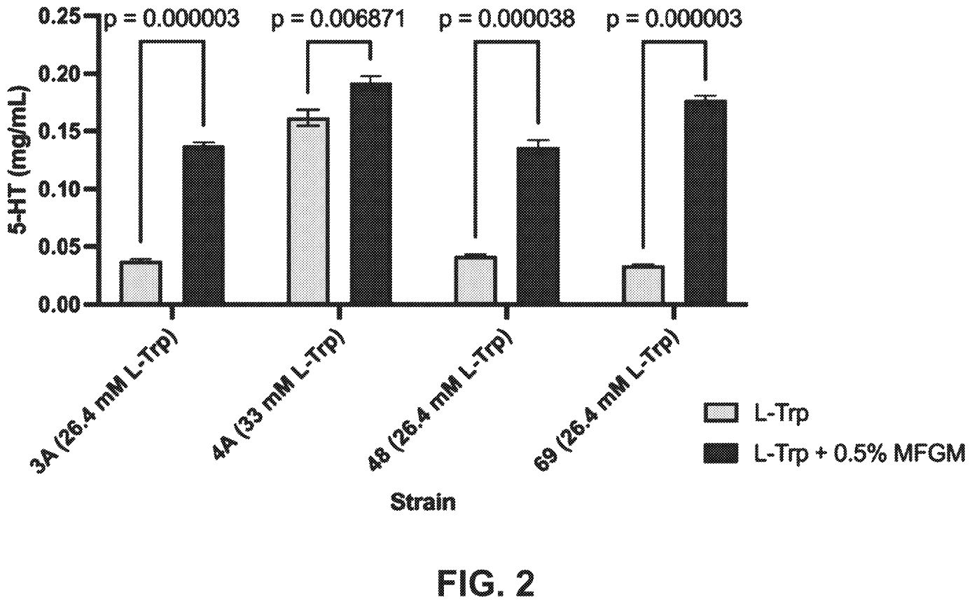

The present work involves the formulation of a food product or nutritional supplement containing a consortia of lactic acid bacteria and milk fat globule membrane to enhance neurotransmitter production in the intestine. It has been demonstrated that these lactic acid bacteria grow well in the presence of milk fat globule membrane in a minimal culture medium (FIG. 1A-FIG. 1D). The capacity for milk fat globule membrane supplementation in the presence of an amino acid precursor (L-Tryptophan) in a minimal bacterial culture medium to promote serotonin production by four lactic acid bacteria strains is also demonstrated (FIG. 2). Up to 5-fold changes in serotonin production are observed. Production of neurotransmitters in the presence of digested milk fat globule membrane and probiotic survival throughout in vitro digestion is also being investigated using in vivo animal models.

This discovery provides a way to enhance the production of neurotransmitters, such as serotonin, in the gut and overcomes the limitations of neurotransmitter oral supplementation. Oral supplementation of neurotransmitters is not feasible as they are generally oxidized during digestive processes and do not reach the intestine. Stimulating neurotransmitter production with milk fat globule membrane and probiotic bacteria is a strategy to enhance mood boosting components that have familiarity with consumers. Milk fat globule membrane-enriched ingredients are derived directly from dairy which has good acceptance amongst consumers and is already being incorporated into infant formula. Probiotic lactic acid bacteria, which are viable through gastrointestinal transit, are also largely accepted and are commonly used as a treatment for gut dysbiosis.

This formulation can be applied to foods and nutritional supplements aimed toward enhancing mood and gut health, as increased serotonin production has been shown to promote satiety and aid in emotional wellbeing. The combination of lactic acid probiotic bacteria, L-Trp, and milk fat globule membrane can be implemented in a wide variety of products including nutritional supplements, non-fermented and fermented foods (i.e., yogurt, cheese, cultured dairy products), frozen desserts (i.e., ice cream), and pet foods. Additionally, this formulation has applications for consumers who experience high stress, such as military personnel, medical professionals, among others.

### Example 2

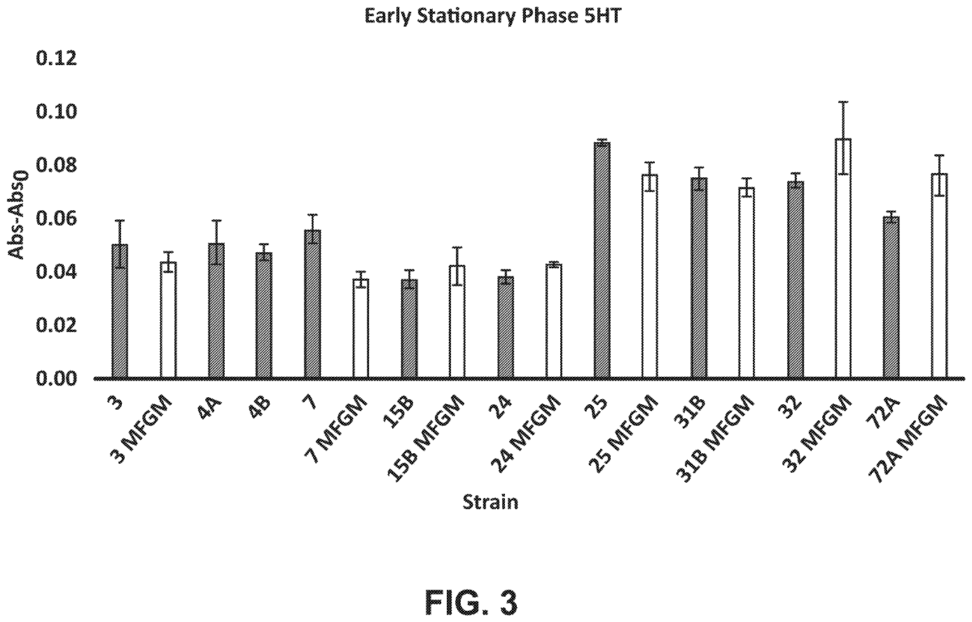

Serotonin (5-HT) production by 10 strains of lactic acid bacteria chosen from a screening of 137 strains in the early stationary phase in the presence of 0.5% milk fat globule membrane (MFGM) and the amino acid precursor, L-Trp, using the minimum bacterial culture media casein glucose broth (CGB) is shown in FIG. 3. Measurements were obtained using the colorimetric Ehrlich's reagent assay using absorbance at 625 nm on a spectrophotometric plate reader. Data are represented as mean±SD with significance values using a Student's t-test (n=6).

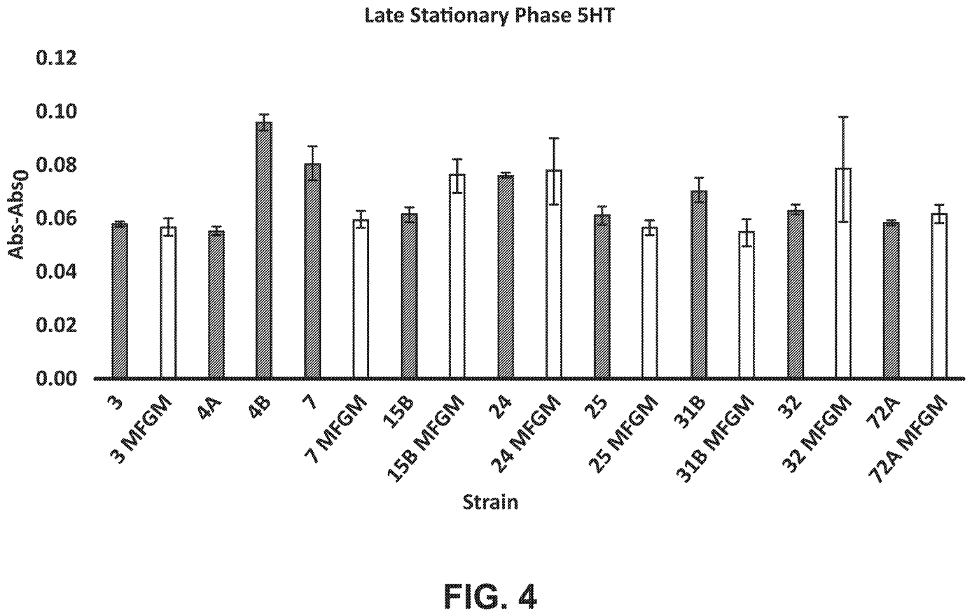

Serotonin (5-HT) production by 10 strains of lactic acid bacteria chosen from a screening of 137 strains in the late stationary phase in the presence of 0.5% milk fat globule membrane (MFGM) and the amino acid precursor, L-Trp, using the minimum bacterial culture media casein glucose broth (CGB) is shown in FIG. 4. Measurements were obtained using the colorimetric Ehrlich's reagent assay using absorbance at 625 nm on a spectrophotometric plate reader. Data are represented as mean±SD with significance values using a Student's t-test (n=6).

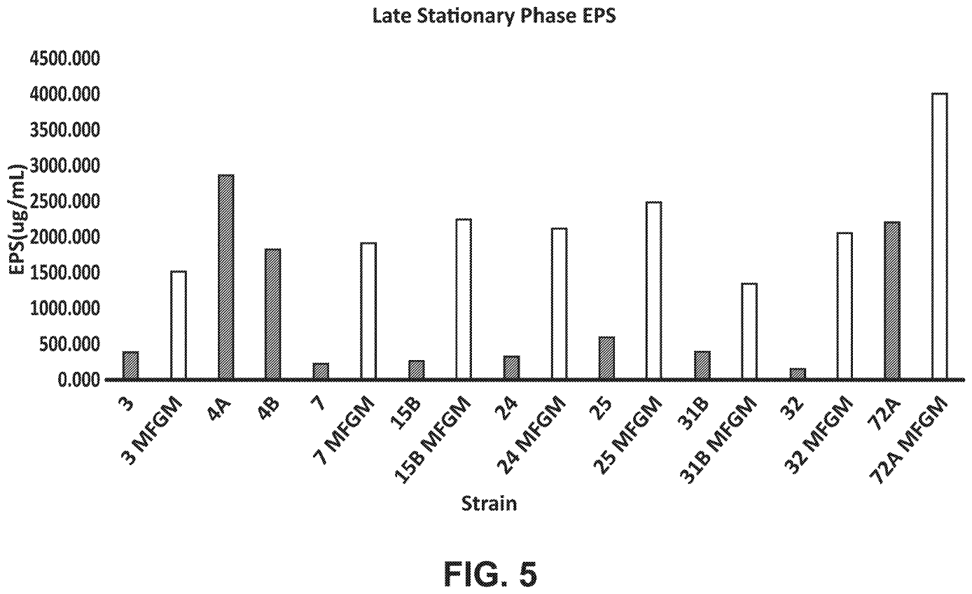

Exopolysaccharide (EPS) production by 10 strains of lactic acid bacteria chosen from a screening of 137 strains in the late stationary phase in the presence of 0.5% milk fat globule membrane (MFGM) and the amino acid precursor, L-Trp, using the minimum bacterial culture media casein glucose broth (CGB) is shown in FIG. 5. Measurements were obtained using the colorimetric phenol sulfuric total sugar assay using absorbance at 480 nm and subtraction of the colorimetric DNSA reducing sugar assay using an absorbance at 540 nm on a spectrophotometric plate reader. Data are represented as mean of the reducing sugars subtracted from the total sugars with significant values using a Student's t-test (n=6).

### Example 3

Described herein is a calming and creamy dark chocolate pudding decorated with a delectable white chocolate shaving topping with active and live cultures.

Described herein is a decadent, calming pudding snack that satiates both the taste buds and the mind through its unique blend of mood-boosting ingredients in a creamy dark chocolate pudding decorated with a delectable white chocolate shaving topping with active and live cultures. Among its ingredients that provide relaxing effects, dutch-process dark cocoa delivers rich color, flavor, antioxidants, and magnesium—an essential mineral for stress homeostasis but usually deficient across adult populations (Pickering et al., 2020). Other calming components in the product include whey-derived milk fat globule membrane (MFGM) ingredient, a bacterial blend, and dairy proteins, including casein. Together these calming components help alleviate stress and improve sleep, which differentiates the product from any other product currently on the market. Unlike other currently available pudding snacks, the product has a higher protein content and provides functional calming ingredients to its consumers.

MFGM has gained significant attention for its positive impacts on cognitive function, brain development, and gut microbiota (Brink and Lonnerdal, 2020). Additionally, MFGM has been shown to improve sleep quality after stress (Thompson et al., 2017). A bacterial culture blend comprising Pediococcus acidilactici and Lactobacillus helveticus aid in the production of serotonin, which can positively impact stress and sleep (Daut and Fonken, 2019; Yong et al., 2020). Additionally, Pediococcus acidilactici is highly proteolytic, which improves the digestibility of caseins and increases the likeliness of bioactive compound production (Rocha-Mendoza et al., 2020). Digestion of caseins facilitates the formation of casomorphins, which help suppress oxidative stress and can have a large impact on calming (Thiruvengadam et al., 2021).

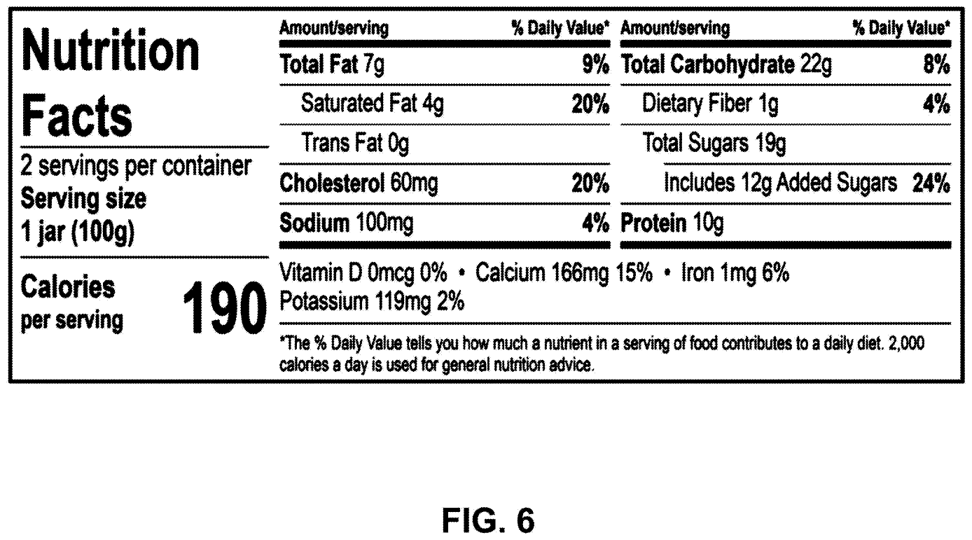

Not only is the product packed with calming ingredients, but in a single 100 g serving, the product contains 10 g of protein (20% of Daily Reference Value), making it an excellent source of protein (FIG. 6). As indicated by the product formulation in Table 1, the product contains 75% dairy ingredients, including whole milk, milk protein concentrate, butter, whey protein isolate, and a whey-derived MFGM ingredient from U.S. dairy farms to help promote dairy consumption in the ever-stressed Gen Z adult population. Reminding its consumers to indulge and relax with dairy as a REAL® and “Cow's milk from the USA” sealed product, the product is the perfect anytime dessert to satiate the mind and body.

TABLE 1
Product formulation.

| Ingredient | Percent (w/w) |
|---|---|
| UHT whole milk | 63.91% |
| Sugar | 12.43% |
| Milk Protein Concentrate (80%) | 6.21% |
| White Chocolate Shaving Topping | 4.71% |
| with live and active cultures | |
| Dutch-process cocoa powder | 3.20% |
| Butter | 2.95% |
| Egg Yolk | 288% |
| Cornstarch | 1.24% |
| Whey Protein Isolate | 1.07% |
| Whey-derived MFGM Ingredient | 0.89% |
| Vanilla extract | 0.53% |
| Total | 100.00% |

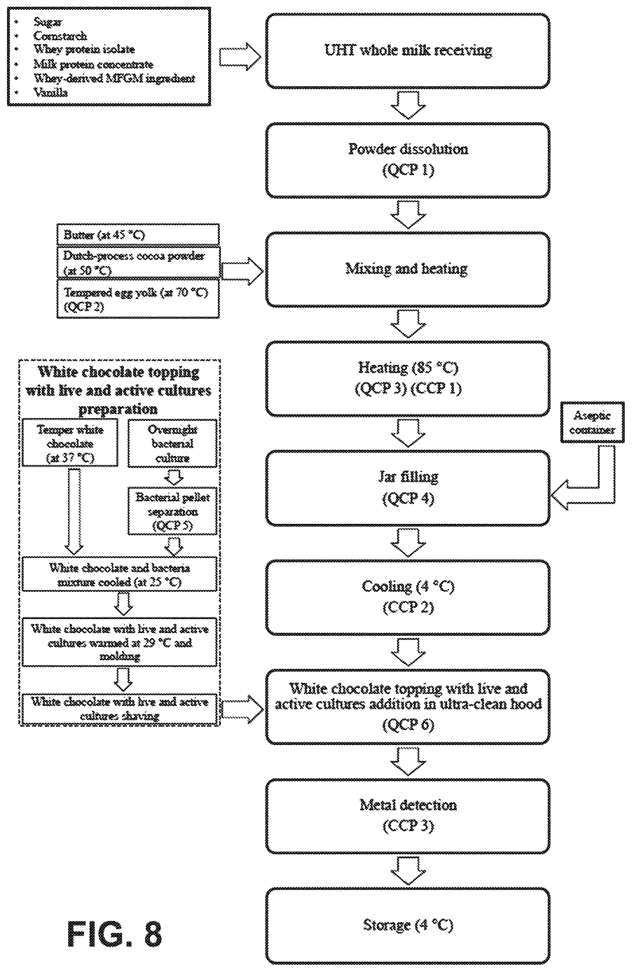

Feasibility Of Production And Process Description. The manufacturing process implements Good Manufacturing Practices, Standard Operating Processes, and Sanitary Standard Operating Procedures as prerequisites. The process flow diagram for the manufacture of the product can be found in FIG. 8. Bulk-packaged UHT whole milk is obtained from a certified local dairy processing facility. Other ingredients are acquired from approved suppliers holding a manufacturer's certificate of assurance (COA). All ingredients are stored at room temperature until their utilization. UHT milk at 25° C. is mixed with sugar, cornstarch, whey protein isolate (WPI), milk protein concentrate (MPC80), whey-derived MFGM ingredient, and vanilla extract in an electric heated kettle with a weep scraper agitator. Powder dissolution (QCP 1) is the first quality control point because no powder agglomeration in the solution can be allowed from this point. After powder dissolution, the mixture is gradually heated to 85° C. During this heating process, ingredients are added as follows: butter at 45° C., cocoa powder at 50° C., and tempered egg yolk at 70° C. (egg yolk tempering is the second quality control point (QCP 2) because of the susceptibility of egg to get curdled, a rapid agitation should be applied to ensure a smooth final product). Cornstarch gelatinization occurs between 80° C. and 85° C. along with whey protein denaturation, which is considered quality control point number 3 (QCP 3) to ensure all water gets trapped and pudding thickness is stable. Reaching 85° C. and holding for 1 min is also considered the first critical control point (CCP 1) because it is required to ensure the product fulfills the thermal treatment requirements to kill harmful microorganisms and extend shelf life. After reaching 85° C., glass jar containers are filled with no less than 96 g of pudding (QCP 4) (to ensure compliance with the label). Each filled jar is sent through an aseptic cooling tunnel (CCP 2) to reach at least 10° C. or below. Subsequently, an ultra-clean hood receives the containers to add the white chocolate shavings topping. The white chocolate topping with live and active cultures is made by growing selected strains of Pediococcus acidilactici and Lactobacillus helveticus overnight in UHT milk, then centrifuging (QCP 5-verification of the only presence of the required cultures is made in each batch growth) and adding the bacterial pellet to white chocolate tempered at 37° C. in an electric chocolate tempering machine. After that, the chocolate is cooled to 25° C., warmed up to 29° C. (to achieve the desired crystalline structure), and poured into molds to solidify. The white chocolate tablets are mechanically shaved, and a topping layer with live and active bacteria (no less than 4 g per container (QCP 6)) is put on top of the pudding. Finally, the container is closed, packed in a 2-pack sleeve, sent through a metal detector (CCP 3), and stored at 4° C. for distribution. Samples from each batch are randomly pulled for quality and microbial analysis. The HACCP plan is shown in Table 5.

TABLE 5
HACCP plan developed for product manufacturing process

| | | (3) | Monitoring | | | | (8) | | (10) |
|---|---|---|---|---|---|---|---|---|---|
| (1) | (2) | Critical | (4) | (5) | (6) | (7) | Corrective | (9) | Record |
| CCP | Hazards | Limits | What | How | Frequency | Who | Actions | Verification | keeping |
| CCP 1 | Biological - | Temperature | Heating | A | Continuous | Processing | Divert and | Check the | Heating |
| Heating | Pathogen survival | to be held | temper- | continuous | Visual | production | destroy | accuracy of | temperature |
| (85 °C) | (Listeria | at 85 °C for | ature | temperature | check | operator | affected | the temperature | log |
| | monocytogenes, | at least | | monitoring | of the | | product. | recording device | Calibration |
| | Salmonella spp., | 15 sec | | device | monitoring | | Determine | against a | log |
| | Escherichia coli., | | | | device | | cause of | certified | |
| | Staphylococcus | | | | every | | deviation | calibration | |
| | aureus, | | | | produced | | and adjust | thermometer; | |
| | Clostridium | | | | batch | | as needed. | Annual | |
| | perfringens, | | | | | | | calibration | |
| | Clostridium | | | | | | | with a | |
| | botulinum, | | | | | | | certified | |
| | Bacillus cereus) | | | | | | | thermometer as | |
| | | | | | | | | recommended. | |
| CCP 2 | Biological - | Chill jar | Cooling | A | Continuous | Processing | Divert and | To check the | Cooling |
| Cooling | Multiplication and | pudding | temper- | continuous | Visual | Production | repeat pass it | accuracy of the | temperature |
| | survival of Listeria | down from | ature | temperature | check | operator | through the | temperature | log |
| | monocytogenes, | 85 °C to | | monitoring | of the | | aseptic | recording | Calibration |
| | Salmonella spp., | 10 °C after | | device | monitoring | | cooling | device against | log |
| | Escherichia coli., | passing it | | | device | | tunnel | a certified | |
| | Staphylococcus | through an | | | every | | | calibration | |
| | aureus, | aseptic | | | produced | | | thermometer; | |
| | Clostridium | cooling | | | jar | | | Annual | |
| | perfringens, | tunnel | | | | | | calibration | |
| | Clostridium | (Record | | | | | | with a | |
| | botulinum, | finish | | | | | | certified | |
| | Bacillus cereus) | temperature). | | | | | | thermometer as | |
| | | | | | | | | recommended. | |
| | | | | | | | | Annual | |
| | | | | | | | | calibration of | |
| | | | | | | | | the aseptic | |
| | | | | | | | | cooling | |
| | | | | | | | | tunnel. | |
| CCP3 | Physical - Metal | No metal | Presence | Metal | Every 2- | Metal | Divert and | Calibrate the | Metal |
| Metal | fragments | fragments | of metal | detector | pack of | detector | destroy the | metal detector | detector |
| detection | introduced | detected in | fragments | | finished | line | affected | to ensure | operation |
| | during | finished | in | | product | operator | product. | pieces 7 mm or | log |
| | processing | product | finished | | | | Identify | greater in | Calibration |
| | | | product | | | | source | length do not | log |
| | | | | | | | of metal | pass. Bi-annual | |
| | | | | | | | fragments | calibration | |
| | | | | | | | | of the metal | |
| | | | | | | | | detector as | |
| | | | | | | | | recommended. | |

Shelf-Life Stability and Safety. During the manufacturing process, the thermal treatment (85° C., CCP1) kills potentially harmful microorganisms, which is followed by aseptic packaging. Additionally, all equipment used during each process step (i.e., mixing, heating, packaging) is thoroughly cleaned and sanitized to prevent contamination and extend shelf life. Quality assurance parameters by means of gel strength analysis (via a texture analyzer), water activity, and pH are regularly tested during the production of the product. Microbiological tests are conducted for Salmonella spp., total plate count, and coliform count to assess for proper aseptic processing and safety and to assure the viability of probiotics during the shelf life of the product.

A shelf-life study was conducted on samples stored at 4° C. over a 4-week period testing every 7 days for physical, microbiological, and sensory properties. Physical properties were measured using a moisture analyzer (Sartorius, Germany), water activity meter (Novasina LabSwift, Switzerland), and pH meter (Mettler Toledo, Columbus, OH). Over the course of 4 weeks, the pudding maintained a stable % moisture content (55.3±3.09), water activity (0.95±0.01), and pH (6.6±0.01). In-house sensory testing relative to a freshly-prepared control indicated no significant changes in the appearance, chocolate flavor intensity, sweetness, and texture.

Microbiological testing was performed by first diluting pudding and white chocolate samples 1:10 in phosphate buffered saline (PBS) buffer and using a Stomacher (Microbiology International, Frederick, MD, USA) to homogenize samples and release microbes from the food matrix. Serial dilutions were plated on total plate count (TPC) agar, deMan Rogosa Sharpe (MRS) agar for Lactobacilli, eosin methylene blue (EMB) agar for the coliform count, and Rose-Bengal Chloramphenicol (RBC) agar for yeasts and molds. The pudding fraction was microbiologically stable over the course of 4 weeks, as <1 CFU/mL was detected in any of the agar (Table 2). However, the microbiological stability of the desirable live and active cultures in the white chocolate topping was more variable, with a 2-log reduction of Lactobacilli from 10⁸ CFU/mL immediately after production (day 0) to 10⁶ CFU/mL by day 21 (Table 3).

TABLE 2
Microbial shelf-life study results for the pudding
fraction of product stored at 4 °C for 28 days.

| | Total | Coliform | Lactobacilli | Yeast & |
|---|---|---|---|---|
| | Plate Count | Count | Count | Mold |
| | TPC Agar | EBM Agar | MRS Agar | RBC Agar |
| Day | CFU/g | CFU/g | CFU/g | CFU/g |
| 0 | <1 CFU/g | <1 CFU/g | <1 CFU/g | <1 CFU/g |
| 7 | <1 CFU/g | <1 CFU/g | <1 CFU/g | <1 CFU/g |
| 14 | <1 CFU/g | <1 CFU/g | <1 CFU/g | <1 CFU/g |
| 21 | <1 CFU/g | <1 CFU/g | <1 CFU/g | <1 CFU/g |
| 28 | <1 CFU/g | <1 CFU/g | <1 CFU/g | <1 CFU/g |

TABLE 3
Microbial shelf-life study results for the white chocolate
topping of product stored at 4 °C for 28 days.

| | Total | Coliform | Lactobacilli | Yeast & |
|---|---|---|---|---|
| | Plate Count | Count | Count | Mold |
| | TPC Agar | EBM Agar | MRS Agar | RBC Agar |
| Day | CFU/g | CFU/g | CFU/g | CFU/g |
| 0 | 10⁹ CFU/g | <1 CFU/g | 10⁸ CFU/g | <1 CFU/g |
| 7 | 10⁸ CFU/g | <1 CFU/g | 10⁸ CFU/g | <1 CFU/g |
| 14 | 10⁷ CFU/g | <1 CFU/g | 10⁷ CFU/g | <1 CFU/g |
| 21 | 10⁶ CFU/g | <1 CFU/g | 10⁶ CFU/g | <1 CFU/g |
| 28 | 10⁴ CFU/g | <1 CFU/g | 10⁴ CFU/g | <1 CFU/g |

The product is expected to last at least 21 days for the maximum benefit, given that there was no significant deterioration in the physicochemical and organoleptic properties at day 21. The “best if used by” date is estimated to be around 21 days after manufacture at refrigeration temperature, with the primary limitation being the survival of the white chocolate live and active cultures. Future efforts to prolong survival of these live and active cultures by means of encapsulation with whey protein may help to extend the “best if used by date” further (de Araujo Etchepare et al., 2020).

Target Market and Marketability. The product is targeted towards the Gen Z adult population who experiences a higher incidence of reported stress compared to other generations. Culminating from factors such as recent current events, uncertainty, and workload; Gen Z reports that their stress levels are negatively affecting their sleep (31%) and eating patterns (28%), resulting in changes in weight (28%) (American Psychological Association, 2020). Furthermore, Gen Z seeks out convenient food options with 40% of Gen Z consumers reporting that convenience is an important factor when making purchasing decisions (American Egg Board, 2020). The solution described herein is three-fold. The product taps into the needs of Gen Z for products that (1) keep them satiated with dairy protein to promote balanced eating habits, (2) encourage stress management and quality sleep, and (3) are convenient, requiring little-to-no preparation.

As Gen Z consumers are shopping both in grocery stores and online via delivery services, the product will first be sold in grocery stores inside and near college campuses that offer in-store and delivery/pick-up options, such as Kroger, Whole Foods/AmazonFresh, and Walmart. The product's marketing team will also establish social media platforms for the brand (i.e., Instagram) and partner with social media “influencers” passionate about living a balanced lifestyle to further reach Gen Z consumers.

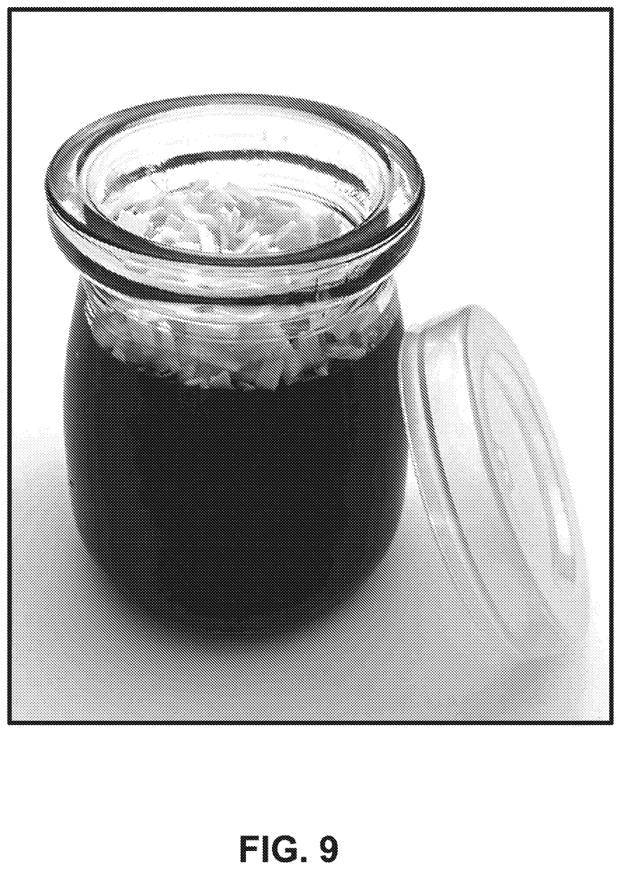

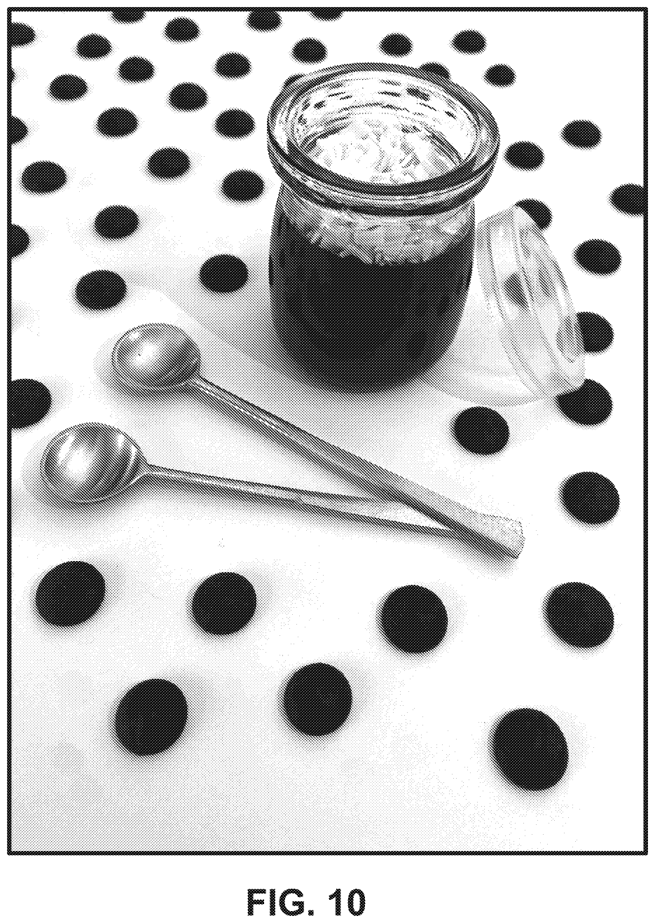

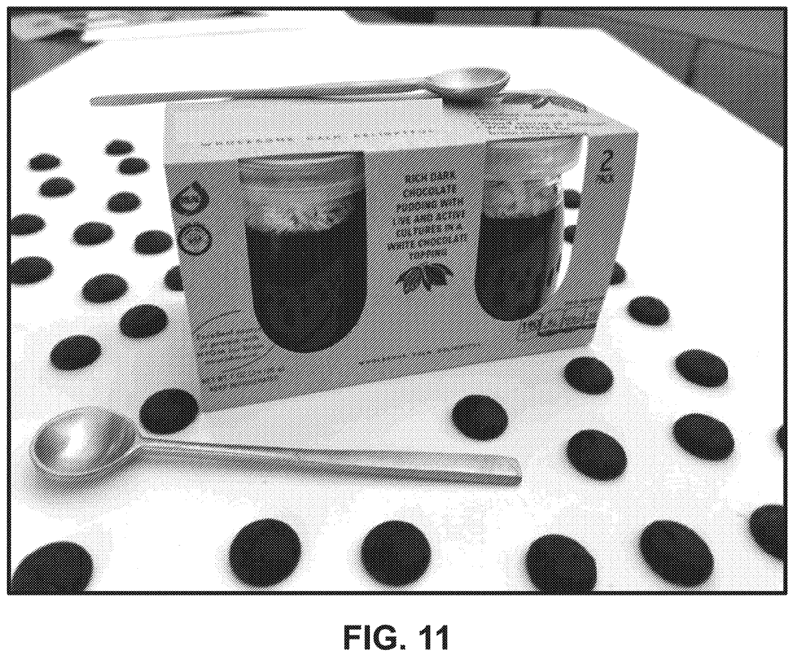

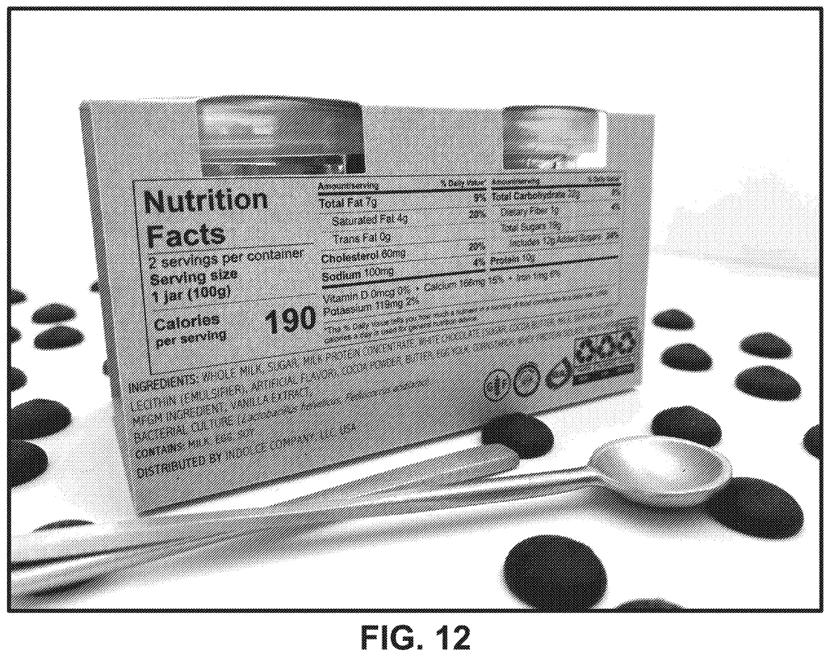

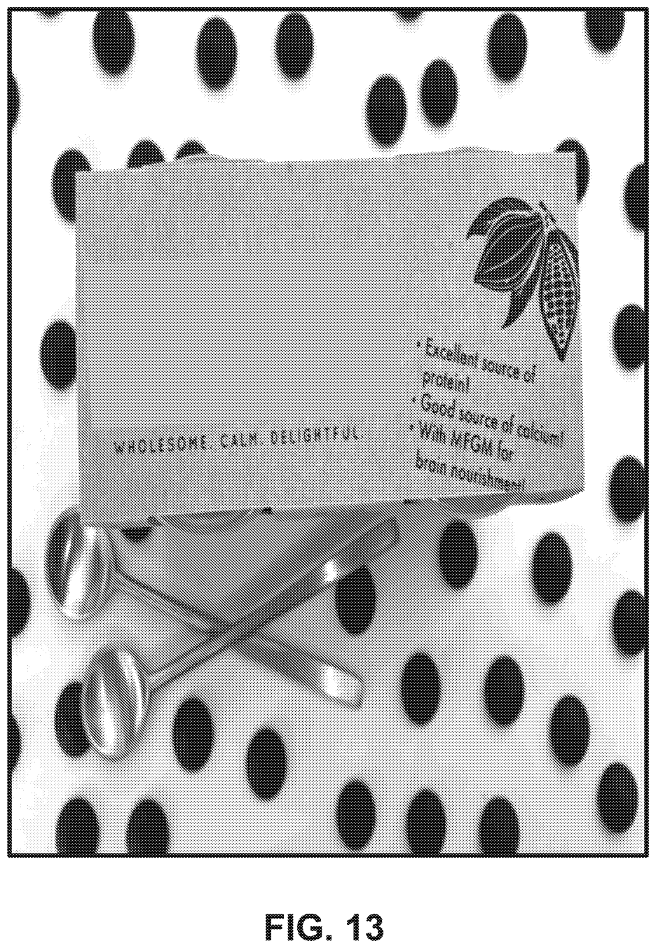

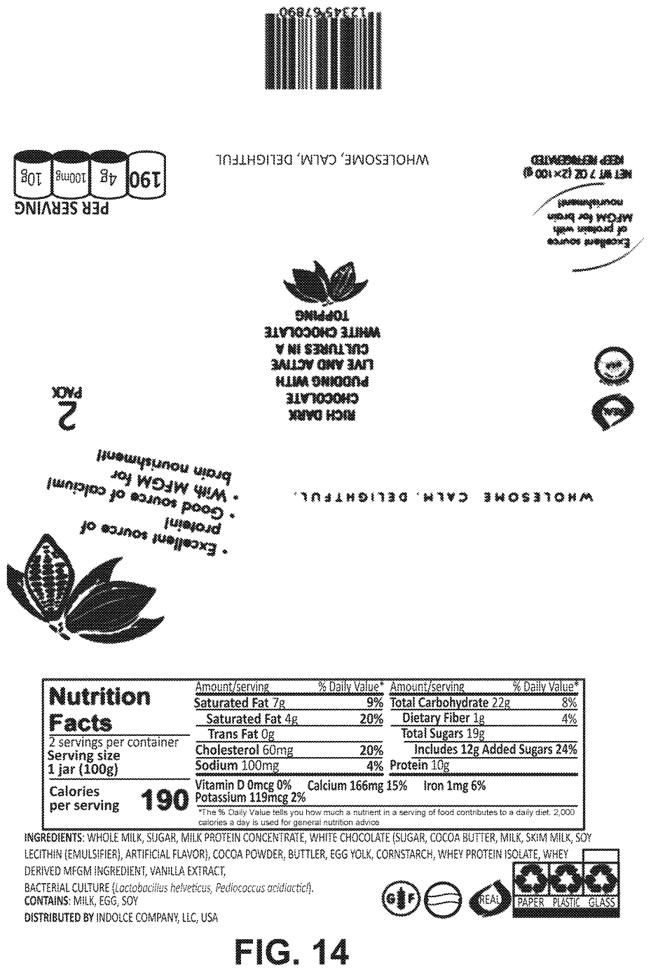

Packaging and Label Claims. With its product placement in the refrigerated dessert and pudding aisle, the product will be positioned as a premium, high-end product using its carefully curated packaging design. As its primary packaging, the product is packaged into 3.5 fl oz glass jars that feature an elegantly curved shape resemblant of a milk bottle and reusable polyethylene lids (FIG. 9-FIG. 13). The secondary packaging (FIG. 14) is composed of folded recycled cardstock that uses two cutout windows to showcase the product directly in a 2-pack. To provide Gen Z consumers with confidence that the product is a quality dairy product, the product has been REAL® Seal certified and sealed with the “Cow's milk from the USA” seal by the U.S. Dairy Export Council to distinguish it as a product that provides satisfying dairy protein and nutrients from U.S. dairy cows. For consumer transparency, the product is also certified as gluten free indicated by the gluten-free seal on the back of the packaging.

For the nutritional claims of the product, Title 21 of the Code of Federal Regulations (CFR) was used as a reference, considering a 2,000-calorie diet. The product is an “excellent source of protein” according to Section 101.54(b)(1) of Title 21 of the CFR as each 100 g serving of the product contains 10 g of protein or 20% of the daily recommended value (DRV). Additionally, a 100 g serving of the product contains 166 mg of calcium, which is 15% of the recommended DRV. According to Section 101.54(c) of Title 21 of the CFR, the product qualifies as a “good source of calcium”. The packaging also uses the statement “with MFGM for brain nourishment” to highlight to the consumer that the functionality of this product extends beyond its nutritional value.

The back of the packaging contains the nutrition facts label, ingredient statement, and a guide for recycling packaging materials. The main materials used to package the product (glass, low density polyethylene, and cardboard) are fully recyclable. In addition, the marketing team will highlight creative ideas on social media to reuse these containers, give them a second life (i.e., storage for beauty products or food products, repurposed candle jars) and extend the product experience after initial consumption. The product team holds high standards of environmental responsibility and prides upon sharing new ideas to upcycle the containers with consumers.

Sensory Evaluation. A group of 32 participants (13 male, 19 female) from Generation Z (age range from 18 to 26 years old) were recruited for voluntary participation in the sensory evaluation of the product. Panelists were screened to exclude any with health conditions that impaired their senses of smell or taste, and individuals with food restrictions (i.e., allergies) that included dairy or egg products. All participants indicated that they consume dairy products daily and sweet snacks weekly. Each panelist was presented with one sample of the product at refrigerated temperature (4° C.) in a 3.5 fl oz glass jar. Sensory tests were conducted in sensory evaluation booths under regular light. Panelists' responses were recorded using the Compusene20 software (Guelph, Ontario). Panelists evaluated concept liking, overall product liking, packaging, appearance, flavor, and texture using a 9-point hedonic scale. Panelists also judged the amount of white chocolate and the intensity of key sensory attributes with a Just-About-Right (JAR) scale, delivered expectations based on the concept initially presented, and purchase intentions for the product.

The results of the sensory evaluation show that the panelists really liked the product concept that was presented to them before being served the product. The container used to serve the product to the panelists was liked moderately, and some comments about it were that it was too narrow, and the solid black lid was unappealing. This feedback was considered while choosing the final packaging for the product.

Nevertheless, when the panelists tasted the product, they liked the overall product very much, with 78% of them giving a score of very much or extreme liking. In the open comments section, the panelists mentioned that they liked the balanced mixture between the dark chocolate in the pudding and the white chocolate in the topping, the smooth and creamy texture of the product, the protein in the product; but what they mentioned the most was how the sweetness of the product was ideal. This is backed by the results of the JAR questions, which show that the product had the right intensity for chocolate flavor intensity (84% of panelists' votes) and sweetness (94% of panelists' votes). Regarding the white chocolate with live and active cultures topping, 84% of the panelists responded that they liked the topping layer from moderately to extremely and 59% of them indicated that amount of topping was just-about-right. But those who responded that there was too little topping still scored their overall product liking from moderately to extremely.

Finally, after evaluating the product, 78% of the panelists indicated that the product exceeded the expectations they had for it based on the product concept that was presented earlier. Regarding the purchase intent for the product, 28% of the panelists responded that they will definitely buy the product, 56% of them would probably buy it, 13% might or might not buy it, and only 3% of them probably will not buy it. In conclusion, the product was well-liked by the panelists in the sensory evaluation and it shows promising potential for commercial production to offer a calming and nourishing sweet dairy treat to the public.

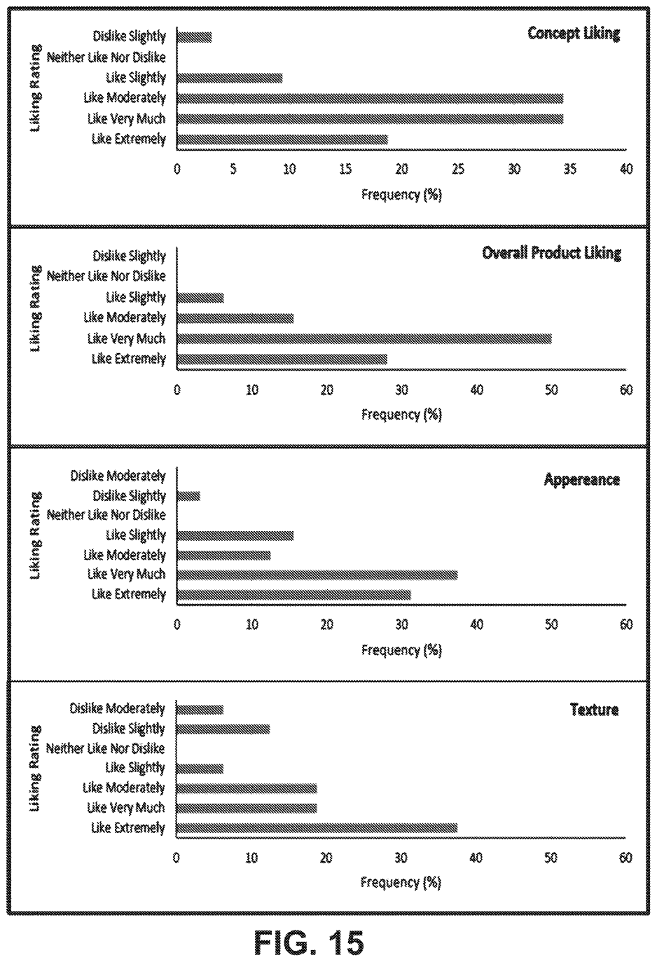

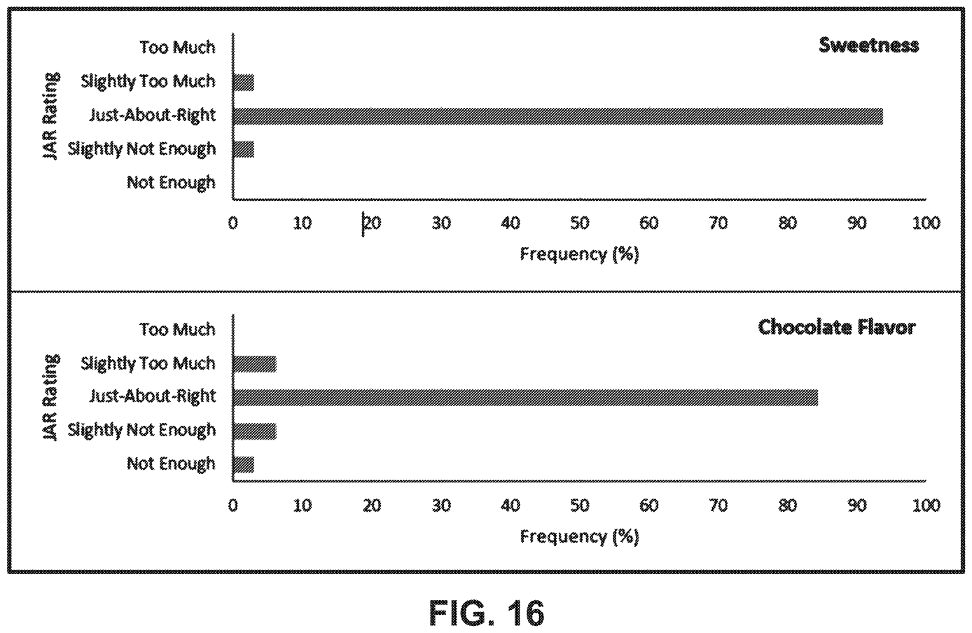

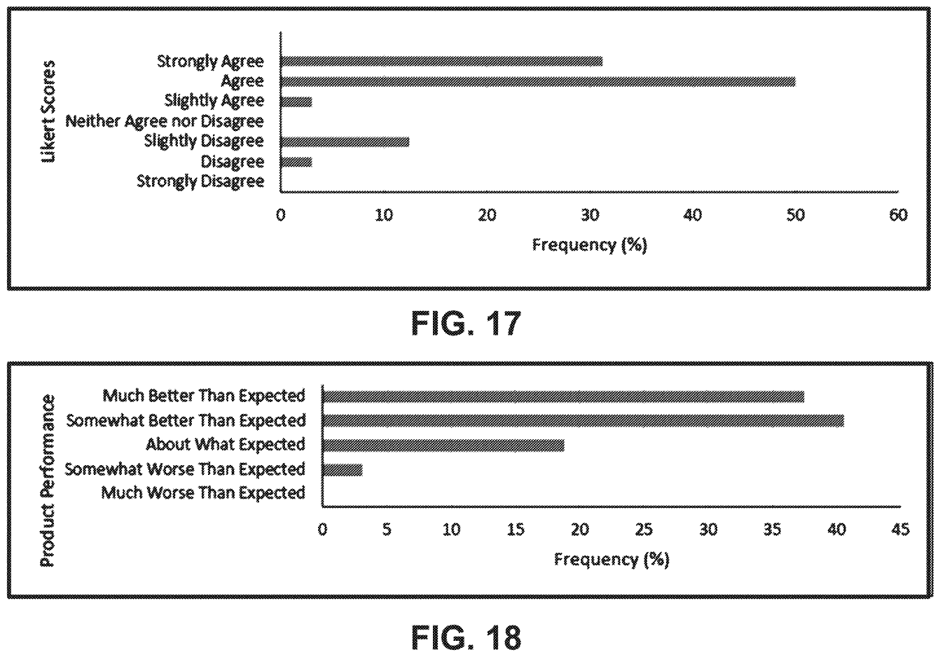

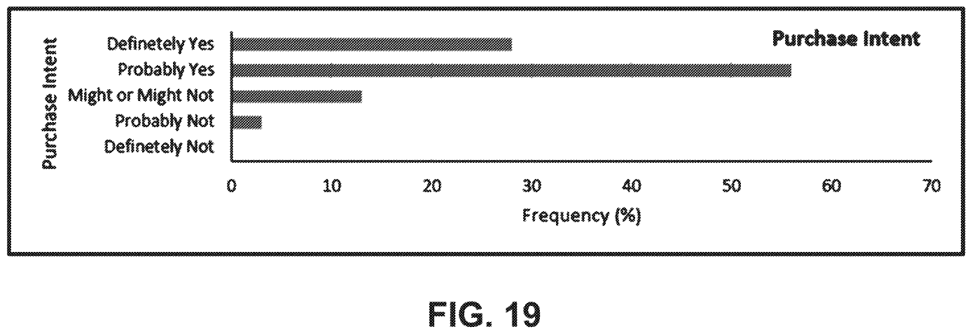

Results of the sensory evaluation are shown in FIG. 15-FIG. 19 and Table 4.

TABLE 4
Penalty analysis results of the Just-About-Right (JAR) evaluation of
product's quantity of white chocolate topping (n = 32).

| | Overall Product Liking | | |
|---|---|---|---|
| Amount of White | Mean Liking | Mean Liking | |
| Chocolate Topping | Score | Score Drop | Consumers (%) |
| Not Enough | 9.00 | 0.74 | 3.1% |
| Slightly Not Enough | 7.57 | −0.69 | 21.9% |
| Just-About-Right | 8.26 | — | 59.4% |
| Slightly Too Much | 7.40 | −0.86 | 15.6% |
| Too Much | — | — | 0.0% |

Dairy Ingredient Functionality. The product contains 75% dairy ingredients, which exceeds de 51% minimum established by this competition's guidelines. Each of these ingredients play critical roles in the texture, flavor, and appearance of the final product largely exceeding the expectation of 51% stipulated in this product development competition. The most abundant dairy ingredient, UHT whole milk, makes up nearly 60% of the product and acts as the main solvent for the powdered ingredients (WPI, MPC80, whey-derived MFGM ingredient, sugar, and cocoa powder). The UHT milk and milk powders contribute most of the product's protein content. Dairy protein powders, along with cornstarch are responsible for thickening and water-binding and giving the pudding a creamy texture. Increasing WPI content helped to reduce egg yolk content in the final product while retaining the smooth consistency of the product. Creaminess and stability are further enhanced by the addition of whey-derived MFGM powder (along with the egg yolk), which have a high phospholipid content to emulsify butter and cocoa fats, and create a desirable mouthfeel in the final product. The fatty acid composition of the butter provides a characteristic flavor of short-chain fatty acids (Pustjens et al., 2017), that after combination with cocoa powder, sugar, and vanilla, give rise to the final flavor and color of the product. The shaved white chocolate topping, which contains minor dairy components, serves as a medium to deliver the live and active cultures (Konar et al., 2018).

Challenges Overcome During the Development of the Product. The product has faced multiple obstacles in delivering a product with high quality and safety standards to the target market. Those challenges are summarized below:

Syneresis: The first challenge that the product faced was syneresis of the pudding as a result of its high moisture content. After only a few days at refrigeration temperature, a watery layer was expelled on the surface of the product. To overcome this technical challenge, milk protein concentrate was increased in the formulation from 4% to 6.21%, and whey protein isolate was added to the formulation (1.07%). Adding these ingredients also helped increase dairy ingredient content in the final formulation, allowing us to reduce cornstarch concentration by 3-fold from 3.75% to 1.24%.

White chocolate topping with live and active cultures addition: The first idea to deliver live and active cultures to our customers was to create a continuous second layer of chocolate over the pudding. However, after collecting data for the preliminary sensory evaluation, some panelists pointed out that the second layer was difficult to break with a spoon. As an initial attempt to resolve this problem, adding heavy cream to the white chocolate to create more of a ganache was attempted, but it created a problem with the consistency during shelf life. The second and final approach was to add the white chocolate with live active cultures as a shaved topping on top of the pudding.

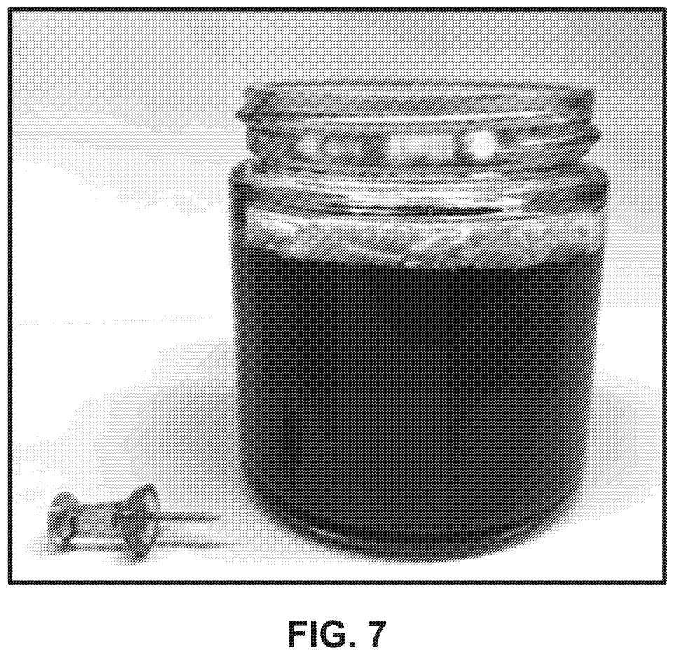

Packaging: A 3.5 fl oz glass jar (presented in FIG. 7) was selected for initial product packaging; however, negative feedback was obtained from sensory analysis because of its shape resemblant of a cosmetic container. As a team, it was decided to replace it with a milk bottle-shaped new container.

Emulsification properties: The product has oil (butter and cocoa fats) and water (fluid milk) phases that need to interact and retain their stability throughout shelf life. At the beginning of the project, the initial formulation contained egg yolk (lecithin) at a 5.62% wt/wt concentration. During the product improvement phase, it was realized that milk phospholipids contained in the whey-derived MFGM ingredient maintained emulsification even when the egg yolk concentration was reduced by almost half (to 2.88%), allowing the proportion of dairy ingredients in the product to be increased.

Link Between Product and DMI Consumer Insights. With recent consumer insights demonstrating a strong interest (nearly 65% of Gen Z adults) and opportunity (projected 20% compound annual growth rate by 2025) for innovative calming foods, the product finds a unique niche in this market opportunity (Dairy Management Inc., 2022). The product taps into this market potential by providing Gen Z with a satiating and creamy, stress-debunking dessert that contains ingredients for relaxation. Pudding is an ideal food matrix to deliver dairy protein and calming ingredients as a dessert. Dessert is a common staple for Gen Z, as almost 90% of Gen Z consumers consider eating dessert multiple times per day and about 50% of consumers have prioritized eating dessert over a meal (Hershey Food Service, 2018). Luckily for Gen Z, the product is not only part of the dessert category, but also a good source of dairy protein, which helps meet daily protein requirements and achieve satiety to improve sleep quality and promote muscle repair (Dairy Management Inc., 2022). Consumer insights have also highlighted the importance of creaminess as a key attribute desired for calming foods (Dairy Management Inc., 2022). The product was developed with this in mind, and its creamy texture will surely leave consumers craving more!

### REFERENCES

American Egg Board. 2020. A New Generation to Feed: Zeroing in on Gen Z's Preferences.
American Psychological Association. 2020. Stress in America.
de Araujo Etchepare, M., G. L. Nunes, B. R. Nicoloso, J. S. Barin, E. M. Moraes Flores, R. de Oliveira Mello, and C. Ragagnin de Menezes. 2020. Improvement of the viability of encapsulated probiotics using whey proteins. LWT 117:108601. doi:10.1016/j.lwt.2019.108601.
Brink, L. R., and B. Lonnerdal. 2020. Milk fat globule membrane: the role of its various components in infant health and development. The Journal of Nutritional Biochemistry 85:108465. doi:10.1016/j.jnutbio.2020.108465.
Dairy Management Inc. 2022. DMI Consumer Insights: Dairy Products for Calming.
Daut, R. A., and L. K. Fonken. 2019. Circadian regulation of depression: A role for serotonin. Frontiers in Neuroendocrinology 54:100746. doi:10.1016/j.yfrne.2019.04.003.
Hershey Food Service. 2018. Gen Z and Millennials Like Hershey Branded Desserts.
Konar, N., I. Palabiyik, O. S. Toker, D. G. Polat, E. Kelleci, H. R. Pirouzian, A. Akcicek, and O. Sagdic. 2018. Conventional and sugar-free probiotic white chocolate: Effect of inulin DP on various quality properties and viability of probiotics. Journal of Functional Foods 43:206-213. doi:10.1016/j.jff.2018.02.016.
Pickering, G., A. Mazur, M. Trousselard, P. Bienkowski, N. Yaltsewa, M. Amessou, L. Noah, and E. Pouteau. 2020. Magnesium Status and Stress: The Vicious Circle Concept Revisited. Nutrients 12:3672. doi:10.3390/nu12123672.
Pustjens, A. M., R. Boerrigter-Eenling, A. H. Koot, M. Rozijn, and S. M. van Ruth. 2017. Characterization of Retail Conventional, Organic, and Grass Full-Fat Butters by Their Fat Contents, Free Fatty Acid Contents, and Triglyceride and Fatty Acid Profiling. Foods 6:26. doi:10.3390/foods6040026.
Rocha-Mendoza, D., E. Kosmerl, G. Miyagusuku-Cruzado, M. M. Giusti, R. Jimenez-Flores, and I. Garcia-Cano. 2020. Growth of lactic acid bacteria in milk phospholipids enhances their adhesion to Caco-2 cells. Journal of Dairy Science 103:7707-7718. doi:10.3168/jds.2020-18271.
Thiruvengadam, M., B. Venkidasamy, P. Thirupathi, I.-M. Chung, and U. Subramanian. 2021. β-Casomorphin: A complete health perspective. Food Chemistry 337:127765. doi:10.1016/j.foodchem.2020.127765.
Thompson, R. S., R. Roller, A. Mika, B. N. Greenwood, R. Knight, M. Chichlowski, B. M. Berg, and M. Fleshner. 2017. Dietary Prebiotics and Bioactive Milk Fractions Improve NREM Sleep, Enhance REM Sleep Rebound and Attenuate the Stress-Induced Decrease in Diurnal Temperature and Gut Microbial Alpha Diversity. Front Behav Neurosci 10. doi:10.3389/fnbeh.2016.00240.
Yong, S. J., T. Tong, J. Chew, and W. L. Lim. 2020. Antidepressive Mechanisms of Probiotics and Their Therapeutic Potential. Front. Neurosci. 13:1361. doi:10.3389/fnins.2019.01361

### EXEMPLARY ASPECTS

In view of the described compositions, devices, systems, and methods, herein below are described certain more particularly described aspects of the inventions. The particularly recited aspects should not, however, be interpreted to have any limiting effect on any different claims containing different or more general teachings described herein or that the “particular” aspects are somehow limited in some way other than the inherent meanings of the language and formulas literally used therein.

Example 1: A composition comprising a probiotic lactic acid bacteria and a milk fat globule membrane.

Example 2: The composition of any examples herein, particularly example 1, wherein the probiotic lactic acid bacteria comprises Pediococcus acidilactici, Lactobacillus helveticus, Limosilactobacillus reuteri, Lacticaseibacillus rhamnosus, or a combination thereof.

Example 3: The composition of any examples herein, particularly example 1 or example 2, wherein the probiotic lactic acid bacteria comprises Pediococcus acidilactici and Lactobacillus helveticus.

Example 4: The composition of any examples herein, particularly examples 1-3, wherein the milk fat globule protein is derived from a milk.

Example 5: The composition of any examples herein, particularly example 4, wherein the milk is obtained from a mammal, such as milk obtained from a cow, goat, horse, donkey, buffalo, camel, or sheep.

Example 6: The composition of any examples herein, particularly examples 1-5, wherein the milk fat globule protein is a whey-derived milk fat globule membrane.

Example 7: The composition of any examples herein, particularly examples 1-6, wherein the composition further comprises any suitable additives, carriers, additional therapeutic agents, bioavailability enhancers, side-effect suppressing components, diluents, buffers, flavoring agents, binders, preservatives or other ingredients that are not detrimental to the efficacy of the composition.

Example 8: The composition of any examples herein, particularly examples 1-7, wherein the composition further comprises an amino acid precursor (e.g., L-Tryptophan).

Example 9: A product comprising the composition of any examples herein, particularly examples 1-8.

Example 10: The product of any examples herein, particularly example 9, wherein the product comprises a nutritional product or supplement.

Example 11: The product of any examples herein, particularly example 9 or example 10, wherein the product comprises a food product, such as a functional food product.

Example 12: The product of any examples herein, particularly examples 9-11, wherein the product comprises any product that normally contains milk or milk solids or milk protein.

Example 13: The product of any examples herein, particularly examples 9-12, wherein the product comprises a dairy product, such as yogurt, cheese, ice cream, pudding, flavored milk, or butter.

Example 14: The product of any examples herein, particularly examples 9-13, wherein the product comprises a non-fermented or a fermented food (e.g., yogurt, cheese, cultured dairy products).

Example 15: The product of any examples herein, particularly examples 9-14, wherein the product comprises a beverage.

Example 16: The product of any examples herein, particularly examples 9-15, wherein the product comprises a dessert product, such as a frozen desserts (e.g., ice cream).

Example 17: The product of any examples herein, particularly examples 9-16, wherein the product comprises an animal food (e.g., pet food, livestock food, and/or laboratory animal food).

Example 18: The composition of any examples herein, particularly examples 1-8 or the product of any examples herein, particularly examples 9-17, wherein the composition or product is for the treatment, prevention, or amelioration of at least one symptom of depression, stress, anxiety, or a depressive or anxiety-related disorder in a subject in need thereof.

Example 19: The composition of any examples herein, particularly examples 1-8 or the product of any examples herein, particularly examples 9-17, wherein the composition or product is for treating serotonin deficiency and/or a disease or disorder related to serotonin deficiency in a subject in need thereof.

Example 20: A method of use of the composition of any examples herein, particularly examples 1-8 or the product of any examples herein, particularly examples 9-17.

Example 21: The method of any examples herein, particularly example 20, wherein the method comprises the treatment, prevention, or amelioration of at least one symptom of depression, stress, anxiety, or a depressive or anxiety-related disorder in a subject in need thereof.

Example 22: The method of any examples herein, particularly example 20 or example 21, wherein the method comprises treating serotonin deficiency and/or a disease or disorder related to serotonin deficiency in a subject in need thereof.

Example 23: The method of any examples herein, particularly examples 20-22, wherein when the composition and/or product is consumed by a subject, the composition and/or product enhances neurotransmitter production in the gastrointestinal tract of the subject.

Example 24: The method of any examples herein, particularly example 23, wherein the neurotransmitter comprises serotonin, dopamine, GABA, or a combination thereof.

Example 25: The method of any examples herein, particularly example 23 or example 24, wherein the neurotransmitter comprises serotonin.

Example 26: The method of any examples herein, particularly examples 20-25, wherein the method results in improved mood, improved gut health, improved satiety, or a combination thereof in the subject.

Example 27: The method of any examples herein, particularly examples 20-26, wherein the subject experiences high stress, such as military personnel and medical professionals.

Example 28: The method of any examples herein, particularly examples 20-27, wherein the subject comprises livestock, a laboratory animal, or a combination thereof.

Example 29: The method of any examples herein, particularly examples 20-27, wherein the subject comprises pigs.

Example 30: A method of making the composition of any examples herein, particularly examples 1-8, or the product of any examples herein, particularly examples 9-17.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A composition comprising a probiotic lactic acid bacteria and a milk fat globule membrane.

2. The composition of claim 1, wherein the probiotic lactic acid bacteria comprises Pediococcus acidilactici, Lactobacillus helveticus, Limosilactobacillus reuteri, Lacticaseibacillus rhamnosus, or a combination thereof.

3. The composition of claim 1, wherein the probiotic lactic acid bacteria comprises Pediococcus acidilactici and Lactobacillus helveticus.

4. The composition of claim 1, wherein the milk fat globule is derived from a milk obtained from a mammal.

5. The composition of claim 4, wherein the milk is obtained from a cow, goat, horse, donkey, buffalo, camel, or sheep.

6. The composition of claim 1, wherein the milk fat globule is a whey-derived milk fat globule membrane.

7. The composition of claim 1, wherein the composition further comprises any suitable additives, carriers, additional therapeutic agents, bioavailability enhancers, side-effect suppressing components, diluents, buffers, flavoring agents, binders, preservatives or other ingredients that are not detrimental to the efficacy of the composition.

8. The composition of claim 1, wherein the composition further comprises an amino acid precursor.

9. A product comprising the composition of claim 1.

10. The product of claim 9, wherein the product comprises a nutritional product or supplement.

11. The product of claim 9, wherein the product comprises a food product.

12. The product of claim 9, wherein the product comprises any product that normally contains milk or milk solids or milk protein.

13. The product of claim 9, wherein the product comprises a dairy product, such as yogurt, cheese, ice cream, pudding, flavored milk, or butter.

14. The product of claim 9, wherein the product comprises a non-fermented or a fermented food.

15. The product of claim 9, wherein the product comprises a beverage.

16. The product of claim 9, wherein the product comprises a dessert product.

17. The product of claim 9, wherein the product comprises an animal food.

18. The composition of claim 1, wherein the composition is for the treatment, prevention, or amelioration of at least one symptom of depression, stress, anxiety, or a depressive or anxiety-related disorder in a subject in need thereof; and/or wherein the composition is for treating serotonin deficiency and/or a disease or disorder related to serotonin deficiency in a subject in need thereof.

19. (canceled)

20. A method of use of the composition of claim 1, wherein the method comprises the treatment, prevention, or amelioration of at least one symptom of depression, stress, anxiety, or a depressive or anxiety-related disorder in a subject in need thereof; and/or wherein the method comprises treating serotonin deficiency and/or a disease or disorder related to serotonin deficiency in a subject in need thereof.

21.-30. (canceled)

31. The composition of claim 8, wherein the amino acid precursor comprises L-Tryptophan.
